(19) United States
(12) Patent Application Publication
(10) Pub. No.: US 2007/0043312 A1
(43) Pub. Date: Feb. 22, 2007

(54) Splint product for fixing fractured bone

(75) Inventor: Jong Han Lee, Gyeongju (KR)

Correspondence Address: SUGHRUE MION, PLLC, 2100 PENNSYLVANIA AVENUE, N.W., SUITE 800, WASHINGTON, DC, 20037, US

(73) Assignee: NEW CAST INDUSTRY CO., LTD.

(21) Appl. No.: 11/205,113

(22) Filed: Aug. 17, 2005

(51) Int. Cl. A61F 5/00

(52) U.S. Cl. 602/6

(57) ABSTRACT

There is provided a splint product which can be mounted on an affected part well without generating wrinkles and which can be conveniently used in a surgical operation. [Solving Means] As a belt-shaped splint sealing package including a support into which a hardener is infiltrated is received in a dispenser in a state where the splint sealing package is folded every predetermined length in an accordion shape. In addition, a belt-shaped accessory cushion accessorily used is received in the dispenser with vertical arrangement in a state where the belt-shaped accessory cushion is folded similarly to the splint sealing package. The splint sealing package and the accessory cushion are taken out from taking-out openings formed in the dispenser and cut by a necessary length for use.

### BACKGROUND OF THE INVENTION

1. Field of the Invention

The present invention relates to a splint product used for fixing a fractured bone or other damaged parts of a human body and more particularly to a free-cut type belt-shaped splint product which can be cut and used by a necessary length in the length direction thereof.

2. Description of the Related Art

Thanks to various advantages such as easy treatment, small weight, easy surgical operation due to reduction in hardening time, water resistance after mounting and hardening, and strength of a splint using a conventional calcined gypsum as a water-hardening synthetic resin, that is, a synthetic resin hardener, a splint containing a synthetic-resin hardener such as an urethane synthetic resin hardener which is hardened in moisture has been widely used. (See Patent Documents 1 and 2)

Such a splint product is made by using fiber fabrics such as thick mesh fabrics having elasticity and air permeability as a support, infiltrating the hardener in the support, disposing a cushion in at least one side thereof, and seal-packing them, and it keeps softness during storage, when it is dipped in water at use, absorbs water, and attached to a predetermined portion of a body, it is hardened and performs a splint function.

Such a kind of splint product includes a free-cut type splint product manufactured in the predetermined length in advance and individually packaged and another free-cut type splint product manufactured in long length in advance and be cut and used in necessary length so that variable length of splint required for using in a fractured bone, etc. is obtained (Patent document 2).

As disclosed in Patent document 2, the long free-cut type splint product is made by seal-packing a splint body consisting of a belt-shaped elastic support into which the hardener is infiltrated and a protection cover made of cushioning fabrics and surrounding the support with a stacked airtight package that is generally called an aluminum pack. Conventionally, the long sealing package with a roll shape wound from one end has come to the market in a state where it is received in a packing box that is a dispenser. A user takes out one end of the roll-shaped splint sealing package from a taking-out opening formed in one sidewall of the dispenser, cuts it by a necessary length for use, seals again the remaining splint sealing package with a separately-provided re-sealing device, and thus prevents the hardening of the splint body when the splint body comes in contact with air.

[Patent Document 1] Japanese Unexamined Patent Application Publication No. 2003-305070

[Patent Document 2] Japanese Patent Publication No. 1993-24696

However, a conventional roll-type splint product disclosed in Patent Document 2 has the following problems.

First, because a long splint sealing package wound in a roll shape has a small diameter as it advances in a central portion of the roll, the airtight package for vacuum packing has many deep and irregular wrinkles in the width direction. Here, because the airtight package has an aluminum layer at its inside, the package has somewhat higher strength and wrinkles generated in the package exert an influence on the splint body received therein and make the support, into which the hardener is infiltrated, have wrinkles and strong winding property. Such a strong winding property or wrinkles makes it difficult to the splint body to be properly mounted in an affected part at a surgical operation. Further, if the package is mounted and hardened in a state having wrinkles, it makes the splint giving bad fit feeling to be formed and exerts a negative influence on a curative effect. In addition, because wrinkles generated at a surgical operation cause a partial weak portion in the hardened splint, the splint may be sometimes broken in a wrinkle portion. Rupture of the splint may make a rupture side to damage a human body, in addition to exert negative impact on an affected part.

Second, when the splint sealing package wound in a roll shape is received in a square box that is a dispenser, the box has large dead space in its four corner portions. That is, capacity of the dispenser is not efficiently used. Thereby, the dispenser for receiving the splint package body becomes greater in size and it is difficult to receive a sufficiently-long splint package in the limited size of the dispenser.

Third, a conventional splint product is made by receiving the splint sealing package in the dispenser, but when the product is actually attached in a human body, it is required to seek elevation of fit feeling, reduction of moisture, prevention of skin abrasion, etc. by interposing a separate cushion between an affected part of a human body and the splint body. For this reason, the conventional splint product should prepare a separate cushion. However, because it is difficult to store the cushion and obtain a good cushion in an attaching place, unnecessary effort and time are required in a surgical operation.

### SUMMARY OF THE INVENTION

The present invention is contrived to solve the above-mentioned problems and it is an object of the present invention to provide a long splint product which does not have the above-mentioned problems.

In order to achieve the above-mentioned object, the present invention provides a splint product for fixing a fractured bone, etc. which has a structure according to any one of Aspects 1 to 9 described below.

According to Aspect 1 of the present invention, there is provided a splint product for fixing a fractured bone, etc., the splint product comprising: a belt-shaped splint sealing package in which a belt-shaped splint body having elasticity is sealed with a sleeve-shaped airtight package over the entire length; a belt-shaped accessory cushion having elasticity which is overlapped and mounted on a surface of the splint body separately from the splint sealing package at the time of mounting the splint body; a dispenser for packing the splint sealing package and the accessory cushion together; and a re-sealing device for re-sealing an opened edge of the airtight package of the remaining splint sealing package after cutting the splint sealing package by a length necessary for use, wherein the splint sealing package and the accessory cushion are vertically arranged in the dispenser in a state where they are folded in an accordion shape or in a sinuous shape with a length corresponding to the depth of the dispenser, and wherein the dispenser has at least one taking-out opening for individually or simultaneously taking out the splint sealing package and the accessory cushion, the at least one taking-out opening being formed in at least one side surface of the dispenser.

According to Aspect 2 of the present invention, the splint body may comprise a support made of elastic and air-permeable fabrics, a hardener infiltrated into the support, a flat tube-shaped cushioning protection cover having a width wider than that of the support, surrounding the entire support, and having elasticity.

According to Aspect 3 of the present invention, the support may be made of a thick sheet of flat knitted fabric.

According to Aspect 4 of the present invention, the support body may be made of rib knitted fabrics having high elasticity whose texture is arranged in the length direction of the splint body.

According to Aspect 5 of the present invention, The protection cover may include two sheets of upper and lower flat fabrics having a width greater than that of the support and may have a flat tube shape formed by inserting the support between the two sheets of flat fabrics and bonding both edges of the two sheets of flat fabrics to each other over the entire length.

According to Aspect 6 of the present invention, the protection cover may be made of an elastic fleece fabric having a nap layer on at least the outer surface.

According to Aspect 7 of the present invention, the accessory cushion may be made of an elastic fleece fabric having a nap layer on at least one surface.

According to Aspect 8 of the present invention, the hardener may be made of a water-hardening synthetic resin.

According to Aspect 9 of the present invention, the dispenser may have two taking-out openings for individually taking out the splint sealing package and the accessory cushion in the upper portion and the lower portion of one side surface of the dispenser to correspond to arrangement of the splint sealing package and the accessory cushion.

In the splint product having the structure according to Aspect 1 of the present invention, it is possible to obtain and use the splint body having a variable length by taking out and cutting the splint sealing package from the dispenser and to store the remaining splint sealing package in a non-hardened stable state for use in next time by sealing again an opening end of a sealing package with a re-sealing device, and obtain an advantage of a free-cut type original long splint product that uses a long splint body without wasting, and obtain the following specific effect.

That is, the splint sealing package is received in a state where it is folded by the length corresponding to the depth of the dispenser in an accordion shape or in a sinuous shape in the dispenser. Therefore, wrinkles of the sealing package body are generated in only the folded portions every predetermined length. This makes it possible to obtain the splint body having a flat shape in which there is no curve or wrinkle and having a predetermined homogeneous length in the length direction, by cutting the splint sealing package at positions around the folded portions for use. Generally, the required length of splint for use in a surgical operation roughly ranges 30 to 50^{cm}, particularly 35 to 45^{cm}. Therefore, it is possible to repeatedly obtain the splint body having the flat shape and which is homogeneous in the length direction by setting the folded portion of the splint sealing package corresponding to the length and receiving the folded splint sealing package in the dispenser. Further, in a case where a longer splint body is required, although the folded portion longitudinally exists in the cut splint sealing package, portions where wrinkles are generated due to folding are limited and the package has wrinkles of approximately straight line in a cross direction, whereby it is possible to correct the wrinkled portion by hand after cutting and homogenize an infiltrated state of the hardener. That is, it is possible to easily correct the splint body so that the splint body has no wrinkled portion in the length direction and the predetermined homogeneous length over the entire length. Therefore, by using the homogeneous splint body having no wrinkle in a surgical operation, a surgical operation can be accurately performed and hardened splint having no wrinkle can be obtained even after hardening, and thus it is possible to solve the above-mentioned problems in the conventional roll-type long splint product.

Further, it is possible to efficiently receive the long splint sealing package in the dispenser, compared to a roll-type splint product. That is, it is possible to efficiently use space within the dispenser without forming large dead space. Therefore, for example, when the splint sealing package having the same length as that of the roll-type splint product is received in the dispenser having equal capacity, it is possible to secure the volume corresponding to the dead space as an effective space for reception and when the accessory cushion is folded in the effective space, it is possible to receive both of the splint sealing package and the accessory cushion while efficiently using the dead space. Therefore, according to the present invention, it is possible to obtain a convenient splint product to receive both of the splint sealing package and the accessory cushion without increasing the size of the dispenser.

Further, because both of the splint sealing package and the accessory cushion can be received and individually or simultaneously taken out from one end of the dispenser, it is possible to simply cut the cushion by a necessary length and interpose and mount it between an affected part and the splint body to be attached to a human body in a wet state by giving water at a surgical operation. Further, by using the cushion, it is possible to obtain advantages such as enhancement of fit feeling when the splint is mounted in an affected part, enhancement of mounting feeling by soft skin contact when the splint is mounted in an affected part, prevention in advance of skin abrasion due to mounting of the splint for a long time, reduction of moisture at the time of mounting the splint body in surgical operation, elevation of a sanitary condition by occasion exchange of the cushion. Therefore, when the splint is mounted by using the cushion, it is possible to obtain high convenience.

In the splint product having the structure according to Aspect 2 of the present invention, in addition to the above-mentioned effect, it is possible to satisfactory mount the splint product in an affected part without wrinkles by covering the entire elastic support in which the hardener is infiltrated into a flat tube-shaped cushioning protection cover, putting it on an affected part, and winding it with bandage while stretching it a little in the length direction and/or the width direction, after dipping it in water and thus giving water to it. Therefore, it is possible to efficiently, simply, and adequately perform a surgical operation. Further, because air permeability required for the splint hardened after a surgical operation can be secured, there is no heat damage and satisfactory mounting feeling is obtained.

In the splint product having the structure according to Aspect 3 of the present invention, because the support is made of a thick sheet of flat knitted textile, compared to a support made of a plurality of stacked fabrics, it has a simplified structure, an advantage in manufacturing, and satisfactory elasticity in the length direction or the width direction, so that it is possible to more easily perform a surgical operation.

In the splint product having the structure according to Aspect 4 of the present invention, it is possible to easily mount the splint having excellent elasticity and no wrinkle as the support is made of knitted fabrics which are made of rib knit whose texture is arranged in the length direction of the splint body.

In the splint product having the structure according to Aspect 5 of the present invention, because the protection cover is made in a flat-tube shape by adhering both edges of two pieces of flat-shape fabrics, compared to the protection cover made by inserting the splint body into the cover knitted in a tube shape in advance, it is possible to simply manufacture the splint body and properly position the support and stably maintain it in the middle in the width direction of the protection cover.

In the splint product having the structure according to Aspect 6 or 7 of the present invention, because the protection cover and the cushion are made of elastic fleece fabric having a nap layer on an outside surface or at least one surface, it is possible to obtain soft skin contact and excellent mounting feeling when a relatively inexpensive material is mounted in the splint.

In the splint product having the structure according to Aspect 8 of the present invention, since the hardener is made of water-hardening synthetic resin, it is possible to provide a splint product having light weight, which can easily handle, having short hardening time and excellent water resistance as described above.

In the splint product having the structure according to Aspect 9 of the present invention, since the taking-out opening for taking out the splint sealing package and a taking-out opening for taking out the accessory cushion are provided in the dispenser, it is possible to conveniently take out the splint sealing package and the accessory cushion by the necessary length for a surgical operation.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view illustrating an exemplary embodiment of the present invention;

FIG. 2 is a perspective view illustrating a splint sealing package and an accessory cushion received in a dispenser.

FIG. 3 is a partially-exploded perspective view of the splint sealing package and the accessory cushion.

FIG. 4 is a transverse cross-sectional view of the splint sealing package.

FIG. 5 is a perspective view illustrating the mounted state of a splint product, where FIG. 5A is a perspective view illustrating the hardened splint body and the accessory cushion separated from an affected part and FIG. 5B is a perspective view illustrating the hardened splint body and the accessory cushion mounted on the affected part.

### DETAILED DESCRIPTION OF THE INVENTION

Hereinafter, a preferred embodiment of the present invention will be described with reference to the accompanying drawings.

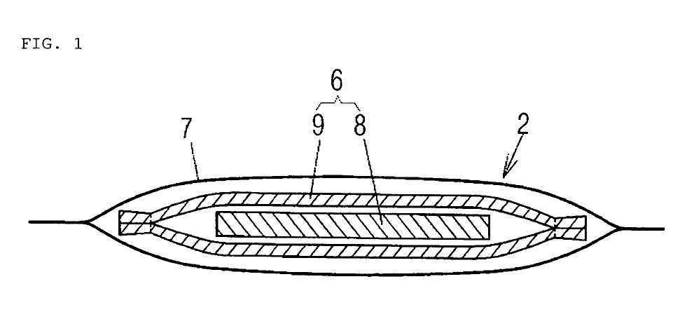

As clearly described in FIGS. 1 through 4, a splint product 1 according to the present invention includes a belt-shaped splint sealing package 2, an accessory cushion 3 which is separate from the package, a dispenser 4 for receiving the package and the cushion, a re-sealing device 5 for using when sealing again after cutting the splint sealing package 2.

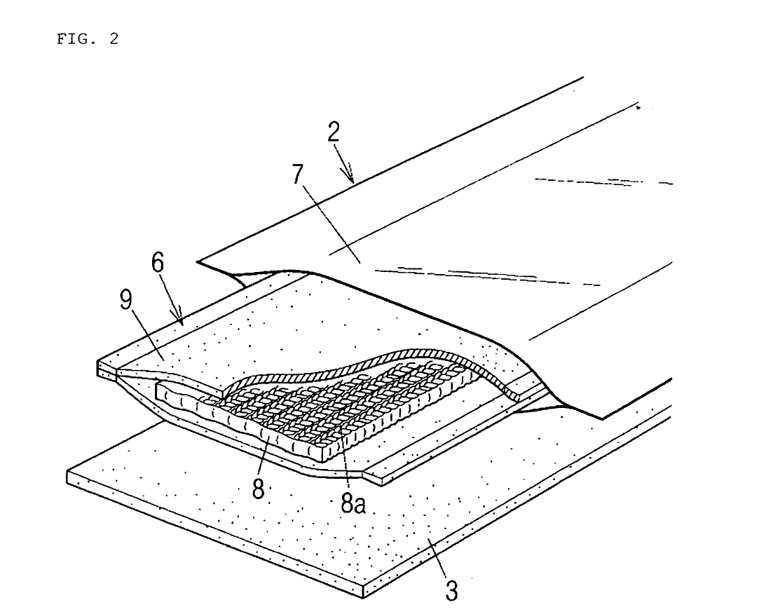

The splint sealing package 2 is formed in a long-belt shape having a width ranging about 15 to 20^{cm} and a length ranging about 400 to 500^{cm}. As shown in FIGS. 1 and 2, the splint sealing package 2 includes a splint body 6 and a sleeve-shaped airtight package 7 that seals and packs the entire splint body 6.

The splint body 6 has a support 8 that is a thick elastic fiber textile made of a sheet of flat knitted fabric therein and a hardener made of, for example, polyurethane water-hardening synthetic resin is infiltrated into the support 8. The entire support 8 is surrounded with a flat-tube shape protection cover 9 that is an elastic cushioning fiber textile having a width greater than that of the support 8.

The support 8 is made of a thick knitted fabric having a thickness of about 5 to 8 mm and a width of about 7.0 to 15.0 mm. Specifically, the support 8 is made of flat-shape knitted fabrics which are made of rib stitches whose texture 8a is arranged in the length direction and has high elasticity in the width direction and the length direction. Further, the support is knitted roughly using knitted yarn consisting of thick polyester yarn, cotton yarn, etc., and thus has many voids between the stitches and has satisfactory air permeability even when the hardener is infiltrated.

The protection cover 9 is made of fleece fabric having a nap layer, that is, soft napped fabric on at least an outside surface and has satisfactory elasticity in the length direction or the width direction. The elasticity may be obtained by using an elastic fiber material and inserting rubber threads into fabrics.

The protection cover 9 is made by inserting the support 8 into which the hardener is infiltrated between upper and lower flat-shape fleece fabric having width wider than the support 8 and made in a flat tube-shape sleeve by adhering both edges protruded from the support 8 with random adhesion connection means. Random means for adhesion, welding, stitching, etc. may be used as the adhesion connection means, but it is preferable to adhere them by heat welding in a narrow line shape not to restrain the protection cover 9 from freely stretching in the length direction. Further, the protection cover 9 may have wider width ranging from about 30 to 50 mm, preferably about 35 to 45 mm than that of the support 8.

The airtight package 7 is composed of three-layer stacked sheets having high airtight which does not pass water. That is, the airtight package 7 is composed of one-piece stacked sheet in which aluminum foil is arranged in the middle and synthetic resin films are stacked on upper and lower surfaces of the aluminum foil. The airtight package 7 has high water impermeability by aluminum foil arranged in the middle and mainly secures strength by synthetic-resin film in the outside, and secures heat welding composition by thermoplastic synthetic-resin film in the inside. However, when the splint body 6 is arranged between two sheets having wide width and a belt shape, both edges of the sealing package 7 are heat welded in a line shape, the sealing package 7 has a flat-tube shape by seal-adhering, and both ends of the sealing package 7 in the length direction are seal-packed to be isolated from air in the length of the entire splint body 6 by seal-adhering. The package may be desirably packaged in vacuum by deflating air from the inside.

The accessory cushion 3 is accessorily used as needed when mounting the splint. The accessory cushion 3 is made of fleece fabric having a dense nap layer on at least one surface and preferably, on both surfaces and has somewhat thicker and higher elasticity than the aforementioned protection cover 9 in the length direction and the width direction.

The accessory cushion 3 has somewhat wider width than the splint body 6, random length, and generally almost equal length with the splint body 6.

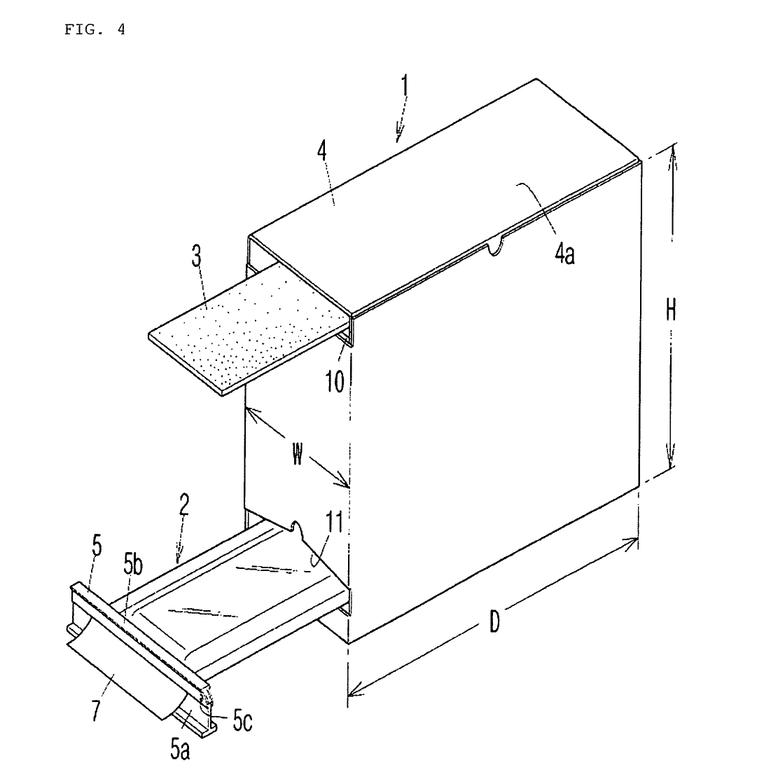

The dispenser 4 is composed of a thick quadrangle paper box, a cardboard box made of paper, a plastic sheet box, etc. As shown in FIG. 4, the width W of the dispenser 4 corresponds to that of the splint sealing package 2 and the height H thereof corresponds to capacity of the splint sealing package 2 and the accessory cushion 3, but length D in the depth direction is an important factor of the present invention. That is, as described later, the splint sealing package 2 is folded in an accordion or sinuous shape to have a predetermined length within the dispenser 4. The splint sealing package 2 has wrinkles in the folded portion but a flat shape between two adjacent folded portions. In order to obtain the flat shape portion having length required as one-unit length of a splint for a surgical operation which is most generally used, it is preferable to set the distance or length between the folded portions within the range from about 30 to 50^{cm}, preferably from about 35 to 45^{cm}. Accordingly, the length D in the depth direction of the dispenser 4 is set to the range from 30 to 50^{cm}, preferably from 35 to 45^{cm} corresponding to the general splint unit length. In the described embodiment, the length D in the depth direction is set to 40^{cm}. Further, its width W and height H are set to 22^{cm} and 35^{cm}, respectively.

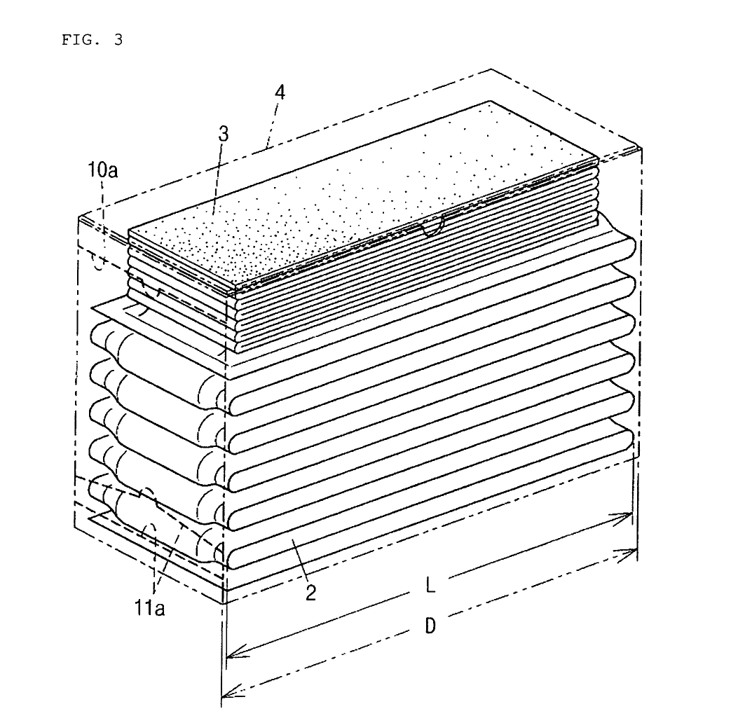

As shown in FIG. 4, two taking-out openings 10 and 11 having a wide slit shape are provided in both ends of the upper and the lower of a front sidewall of the dispenser 4. As shown in FIG. 3, the taking-out openings 10 and 11 are closed at transport and storage of the splint product 1 and opened by removing cutting portions along cutting lines 10a and 11a such as perforation when using the splint. Further, a flap 4a (see FIG. 4) in the top side of the dispenser 4 may be opened or closed and accessories, for example, gloves for a surgical operation, a user's manual, a re-sealing device, a scissor for cutting, etc. received in an upper portion thereof may be put or taken out by opening the flap 4a.

However, as described above, the splint sealing package 2 and the accessory cushion 3 have, for example, long length of 450^{cm}, but as shown in FIG. 3, they are sequentially folded in an accordion or sinuous shape in the length (L) corresponding to the depth (D) of the dispenser 4 in the opposite direction to each other and are received in the dispenser 4. In the embodiment, the splint sealing package 2 and the accessory cushion 3 are received in the lower side and the upper side, respectively, but they may be arranged in opposite direction. However, when they are arranged in the lateral direction, side edges of the splint sealing package 2 may undesirably generate wrinkles by pressing itself due to weight thereof.

The re-sealing device 5 is a device required for sealing to reuse the remaining splint sealing package 2 after cutting and using the splint sealing package 2 in necessary length. Therefore, the re-sealing device 5 may seal again the opened end of the cut airtight package 7, and a structure, form, etc. thereof is not limited. According to the embodiment, as shown in FIG. 4, the clamp-shaped re-sealing device 5 has constriction members 5a and 5b which is made of synthetic resin and which is attached to be opened and closed at a pivot 5c. The re-sealing device 5 has a structure in which a corresponding concave portion of the constriction member 5b is fitted in a flat portion of the constriction member 5a and an end edge of the airtight package 7 is inserted between the constriction members 5a and 5b and completely sealed as a cross-section of the package 7 is made in a U shape.

The splint product is used as follows.

First, as described in FIG. 4, the splint sealing package 2 is taken out and cut with a scissor by necessary length from the lower taking-out opening 11 of the dispenser 4. After the end of the splint body 6 in the remaining sealing package body 2 is folded and received into the airtight package 7 by several centimeters, the opening end of the airtight package 7 is sealed again by the re-sealing device 5 for use in next time. The cut position of the splint sealing package 2 corresponds to the splint length required for a surgical operation and although the position is not always specified, it is preferable that it is set in a folded line or a neighboring portion of the folded line of the splint sealing package 2 folded in an accordion shape. Accordingly, the splint body 6 has no wrinkle due to folding the splint body in a predetermined cut length. Further, the accessory cushion 3 is taken out from the upper taking-out opening 10 of the dispenser 4 and is cut by a somewhat greater length than the cut length of the splint sealing package 2.

After being taken out from the airtight package 7, the splint body 6 is lightly stretched by both hands in the length direction, folded in suitable length at once or several times as needed, dipped in water under temperature ranging from about 18 to 23° C. that is room temperature, and lightly fingered for time ranging from about 5 to 12 seconds so that water is permeated in the entire splint body 6.

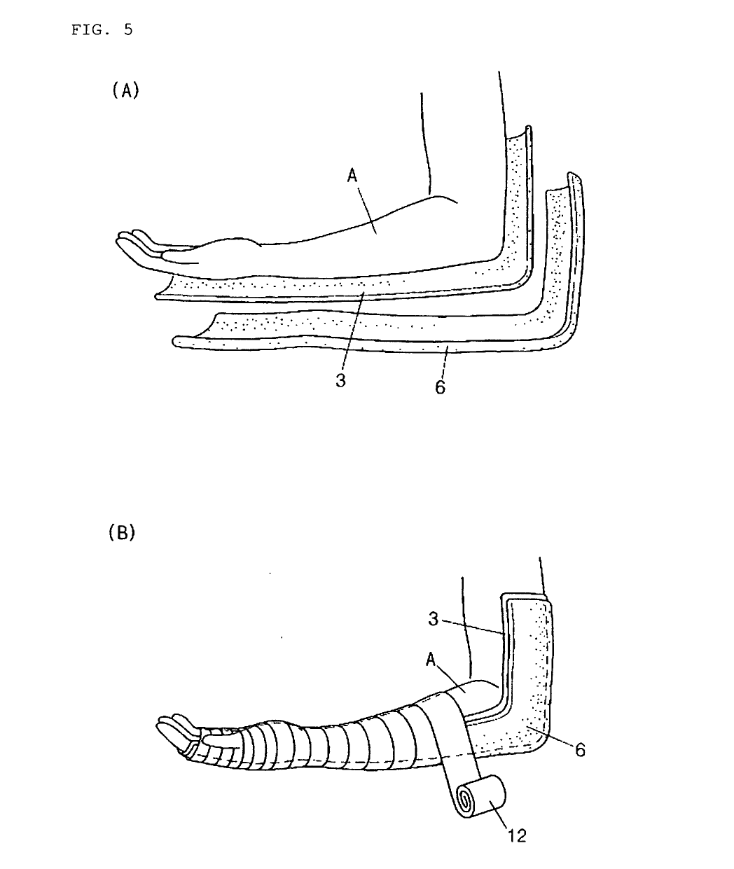

Then, after water in the splint body 6 is removed by softly pressing the splint body 6 by both hands and water on the surface thereof is removed by inserting it between a dry towel, to mount the splint body 6 and the accessory cushion 3 in an affected part is completed by speedily winding a bandage 12 from the outside as shown in FIG. 5B while confirming that wrinkle is not generated after stacking the accessory cushion 3 on one surface of the splint body 6 and slightly stretching both in an affected part A to be fit in appearance of an affected part A.

After mounting the splint body 6 in an affected part, as the hardener infiltrated into the support 8 reacts with water and is hardened for several minutes, as shown in FIG. 5A, the splint is molded depending on appearance of the affected part and fixed to the affected part.

The present invention is not limited to the embodiments. The present invention may be modified in various forms without departing from the descriptions of Aspects 1 to 9 of the present invention.

For example, the support 8 having a tube-shape which made of mesh knitted fabrics used in a conventional splint product may be used in a flat shape. Further, the protection cover 9 and the accessory cushion 3 are not limited to fleece fabric, but they may use other knitted fabric or non woven fabric, etc. if the fabrics have elasticity, cushion property, good skin contact feeling, and air permeability.

Further, as in the embodiment, it is preferable that the taking-out openings 10 and 11 of the dispenser 4 are provided in the upper and lower portions and the splint sealing package 2 and the accessory cushion 3 are individually taken out from the respective taking-out openings, but only one taking-out opening may be provided in the middle of a sidewall or one or two taking-out opening may be provided in a top wall where there is a top flap, and the splint sealing package 2 and the accessory cushion 3 may be simultaneously taken out from the taking-out opening.

The present invention is used to fix a fractured bone and a damaged part of a body from the outside for treating relevant parts in a surgical treatment field.

1. A splint product for fixing a fractured bone, etc., the splint product comprising:
a belt-shaped splint sealing package in which a belt-shaped splint body having elasticity is sealed with a sleeve-shaped airtight package over the entire length;
a belt-shaped accessory cushion having elasticity which is separate from the splint sealing package and is overlapped and mounted on a surface of the splint body at the time of mounting the splint body;
a dispenser for packing the splint sealing package and the accessory cushion together; and
a re-sealing device for re-sealing an opened edge of the airtight package of the remaining splint sealing package after cutting the splint sealing package by a length necessary for use,
wherein the splint sealing package and the accessory cushion are vertically arranged in the dispenser in a state where they are folded in an accordion shape or in a sinuous shape with a length corresponding to the depth of the dispenser, and
wherein the dispenser has at least one taking-out opening for individually or simultaneously taking out the splint sealing package and the accessory cushion, the at least one taking-out opening being formed in at least one side surface of the dispenser.

2. The splint product according to claim 1, wherein the splint body comprises a support made of elastic and air-permeable fabrics, a hardener infiltrated into the support, a flat tube-shaped cushioning protection cover having a width wider than that of the support, surrounding the entire support, and having elasticity.

3. The splint product according to claim 2, wherein the support is made of a thick sheet of flat knitted fabric.

4. The splint product according to claim 2, wherein the support body is made of rib knitted fabrics having high elasticity whose texture is arranged in the length direction of the splint body.

5. The splint product according to claim 2, wherein the protection cover includes two sheets of upper and lower flat fabrics having a width greater than that of the support and has a flat tube shape formed by inserting the support between the two sheets of flat fabrics and bonding both edges of the two sheets of flat fabrics to each other over the entire length.

6. The splint product according to claim 2, wherein the protection cover is made of an elastic fleece fabric having a nap layer on at least the outer surface.

7. The splint product according to claim 2, wherein the accessory cushion is made of an elastic fleece fabric having a nap layer on at least one surface.

8. The splint product according to claim 2, wherein the hardener is made of a water-hardening synthetic resin.

9. The splint product according to claim 1, wherein the dispenser has two taking-out openings for individually taking out the splint sealing package and the accessory cushion in the upper portion and the lower portion of one side surface of the dispenser to correspond to arrangement of the splint sealing package and the accessory cushion.
